(19) United States
(12) Patent Application Publication
(10) Pub. No.: US 2025/0099714 A1
(43) Pub. Date: Mar. 27, 2025

(54) CLICK-LOCK REMOVAL SHEATH

(71) Applicant: Boston Scientific Scimed, Inc., Maple Grove, MN (US)

(72) Inventors: Daniel Presteng, Corcoran, MN (US); Lauren Ann Sandy, Minneapolis, MN (US); Benjamin Jerome Bidne, Loretto, MN (US)

(73) Assignee: Boston Scientific Scimed, Inc., Maple Grove, MN (US)

(21) Appl. No.: 18/891,002

(22) Filed: Sep. 20, 2024

Related U.S. Application Data
(60) Provisional application No. 63/540,349, filed on Sep. 25, 2023.

(51) Int. Cl. A61M 25/00; A61M 25/06; A61M 39/06

(52) U.S. Cl. CPC A61M 25/0097; A61M 25/0023; A61M 25/0068; A61M 25/0668; A61M 39/06; A61M 2039/062; A61M 2039/0626; A61M 2039/064

(57) ABSTRACT

A hub and sheath assembly configured to be releasably disposed over a medical device. The hub and sheath assembly may comprise a valve hub including a first body portion, a second body portion, and a lumen extending therethrough and an elongate shaft including a lumen and a seam. The lumen and the seam may extend from a proximal end to a distal end of the elongate shaft. The elongate shaft may be releasably coupled to the valve hub. The seam may be movable from an interlocked configuration configured to hold the elongate shaft in a generally tubular configuration and an open configuration defining an opening extending along a length of elongate shaft.

### CROSS-REFERENCE TO RELATED APPLICATIONS

This application claims the benefit of priority under 35 U.S.C. §119 of U.S. Provisional Application No. 63/540,349, filed Sep. 25, 2023, the entire disclosure of which is hereby incorporated by reference.

### TECHNICAL FIELD

The present disclosure pertains to sheaths for removing and/or delivering intravascular medical devices. More specifically, the present disclosure relates to a sheath which may be disposed over an in-situ medical device.

### BACKGROUND

In various procedures for delivering intravascular medical devices, a sheath is inserted into a blood vessel of a patient, for example a femoral artery, and one or more medical devices may be advanced through the sheath and into the patient's vasculature. In various instances, the medical devices include catheters or other devices, such as a blood pump. The sheath may remain in place while the medical device is within the body to facilitate removal of the medical device when the procedure is complete. A device that is in use for an extended period of time (e.g., multiple days) may require minimum disruption to blood flow to avoid ischemia. In some cases, a sheath may reduce blood flow while it remains in the body. Thus, there is a need for improved sheaths for introduction and/or removal of medical devices that minimally disrupt blood flow while the medical device is in use.

### BRIEF SUMMARY

This disclosure provides design, material, manufacturing method, and use alternatives for medical devices, including introducer and/or removal sheaths.

In a first example, a hub and sheath assembly may comprise a valve hub including a first body portion, a second body portion, and a lumen extending therethrough, and an elongate shaft including a lumen and a seam. The lumen and the seam may extend from a proximal end to a distal end of the elongate shaft. The elongate shaft may be releasably coupled to the valve hub. The seam may be movable from an interlocked configuration configured to hold the elongate shaft in a generally tubular configuration and an open configuration defining an opening extending along a length of elongate shaft.

Alternatively or additionally to any of the examples above, in another example, the seam may include a first connection member and a second connection member.

Alternatively or additionally to any of the examples above, in another example, the first connection member and the second connection member may be configured to form a releasable interlock.

Alternatively or additionally to any of the examples above, in another example, the first connection member may comprise a first leg, a second leg extending generally parallel to the first leg, a curved region connecting the first leg and the second leg, and a gap between the first leg and the second leg and the second connection member may comprise a first leg, a second leg extending generally parallel to the first leg, a curved region connecting the first leg and the second leg, and a gap between the first leg and the second leg.

Alternatively or additionally to any of the examples above, in another example, when the seam is in the interlocked configuration, the first leg of the first connection member may be disposed within the gap of the second connection member and the first leg of the second connection member may be disposed within the gap of the first connection member.

Alternatively or additionally to any of the examples above, in another example, a distal end region of the elongate shaft may taper towards the distal end thereof.

Alternatively or additionally to any of the examples above, in another example, a distal end region of the elongate shaft may include a plurality of flaps. The plurality of flaps may be configured to radially expand.

Alternatively or additionally to any of the examples above, in another example, the first body portion may be releasably coupled to the second body portion.

Alternatively or additionally to any of the examples above, in another example, the hub and sheath assembly may further comprise a first sealing member disposed within the lumen of the hub.

Alternatively or additionally to any of the examples above, in another example, the hub and sheath assembly may further a second sealing member disposed within the lumen of the hub.

Alternatively or additionally to any of the examples above, in another example, the first sealing member may comprise a slit extending through a thickness thereof.

Alternatively or additionally to any of the examples above, in another example, the second sealing member may comprise a slit extending through a thickness thereof. The slit of the second sealing member may extend generally orthogonal to the slit of the first sealing member.

Alternatively or additionally to any of the examples above, in another example, the first sealing member may be coupled to the first body portion and the second sealing member may be coupled to the second body portion.

Alternatively or additionally to any of the examples above, in another example, the first body portion may include one or more pins extending from a connection surface thereof and the second body portion may include one or more apertures extending into the second body portion from a connection surface thereof.

Alternatively or additionally to any of the examples above, in another example, the one or more pins may be configured to be releasably coupled with the one or more apertures.

In another example, a hub and sheath assembly may comprise a valve hub and an elongate shaft. The valve hub may comprise a first body portion, a second body portion releasably coupled to the first body portion, a lumen extending from a proximal end to a distal end of the valve hub, and a first sealing member disposed within the lumen of the valve hub The elongate shaft may have a lumen and a seam including a first connection member and a second connection member. The lumen and the seam may extend from a proximal end to a distal end of the elongate shaft. The elongate shaft may be releasably coupled to the valve hub. The seam may be movable from an interlocked configuration configured to hold the elongate shaft in a generally tubular configuration and an open configuration defining an opening extending along a length of elongate shaft.

Alternatively or additionally to any of the examples above, in another example, a distal end region of the elongate shaft may taper towards the distal end thereof.

In another example, a method for removing a medical device from a body lumen may comprise positioning an elongate sheath having an opening extending along a length thereof over a portion of the medical device exterior to the body lumen, securing a first connection member and a second connection member of the elongate sheath along the length of the elongate sheath to form a tubular member, coupling a hemostasis valve hub to a proximal end region of the elongate sheath to form a valve hub and sheath assembly, distally advancing the hemostasis valve hub and sheath assembly over the medical device and into the body lumen, and proximally retracting the medical device from the body lumen through the hemostasis valve hub and sheath assembly.

Alternatively or additionally to any of the examples above, in another example, coupling the hemostasis valve hub to the proximal end region of the elongate sheath may comprise coupling a first body portion of the hemostasis valve hub to a second body portion of the hemostasis valve hub over the medical device at a location proximal to a proximal end of the sheath and distally advancing the hemostasis valve hub over the proximal end of the sheath.

Alternatively or additionally to any of the examples above, in another example, the first connection member and the second connection member may form a hemostatic seal.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a side view of an introducer sheath extending into a blood vessel;

FIG. 2 is a cross-sectional view of a portion of the introducer sheath of FIG. 1 inserted into a blood vessel, and a medical device inserted into the introducer sheath;

FIG. 3 is a proximal perspective view of a portion an illustrative sheath;

FIG. 4 is a distal perspective view of the illustrative sheath of FIG. 3;

FIG. 5 is a distal perspective view of the illustrative sheath having an alternative distal end region;

FIG. 6 is an end view of the illustrative sheath of FIG. 3 in an open configuration;

FIG. 7 is a perspective view of an illustrative removable hemostasis valve;

FIG. 8 is a cross-sectional view of the illustrative hub of FIG. 7 taken at line 8-8 of FIG. 7;

FIG. 9 is a cross-sectional view of the illustrative hub of FIG. 7 taken at line 9-9 of FIG. 7;

FIG. 10 is a cross-sectional view of the illustrative hub of FIG. 7 taken at line 10-10 of FIG. 7; and

FIG. 11 is a schematic view of the illustrative sheath and hub disposed over a medical device prior to removal of the medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Some medical devices may extend percutaneously into the body and remain in position for an extended period of time (e.g., hours, days, weeks, etc.). An introducer sheath may be used to facilitate introduction of the medical device into the vasculature. For example, a sheath may be introduced into the femoral artery and one or more medical devices may be advanced through the sheath and into the patient's vasculature. In some embodiments, the medical device may include catheters or other devices, such as, but not limited to, a blood pump. The introducer sheath may remain within the body while the medical device remains in use with a hemostasis valve hub assembly helping to facilitate insertion of the medical device as well as helping to prevent blood from leaking during the medical procedure. When the medical procedure is complete, or the medical device is no longer needed, the introducer sheath may facilitate removal of the medical device. It is desirable for a medical device that is in use for a prolonged period of time (e.g., more than a day) to minimally disrupt blood flow to avoid critical limb ischemia. However, an introducer sheath may reduce blood flow for as long as it remains within the body increasing the risk of ischemia. Further, maintaining a clean and sterile sheath for extended use may present problems for the patient and/or the clinician. Some embodiments of the present disclosure are directed towards a sheath, such as, but not limited to, a removal sheath, that can be positioned over a medical device that is already in place within the body thus minimizing the impact of the sheath on blood flow.

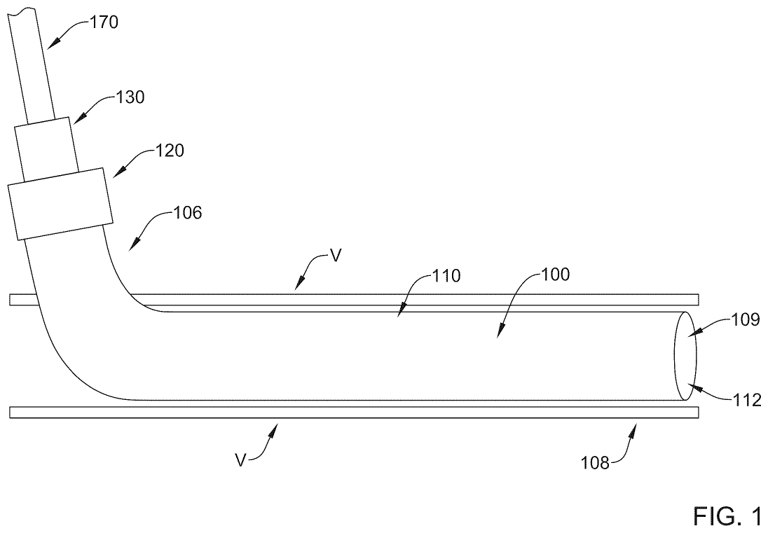

FIG. 1 illustrates a side view of an introducer sheath 100 inserted at least partially into a blood vessel V, shown in cross-section. In some embodiments, the introducer sheath 100 may be used for facilitating the passage of various medical devices, such as a catheter or a blood pump, as will be described further herein, through the introducer sheath 100 and into the blood vessel V. The introducer sheath 100 includes a proximal end region 106 proximate a proximal end of the introducer sheath 100 and a distal end region 108 proximate a distal end of the introducer sheath 100 that is opposite the proximal end region 106. A body portion 110 of the introducer sheath 100 extends between the proximal end region 106 and the distal end region 108, and the body portion 110 defines a lumen 112 of the introducer sheath 100. The introducer sheath 100 includes a proximal opening (not shown) adjacent the proximal end region 106 and a distal opening 109 adjacent the distal end region 108, with the lumen 112 extending from the proximal opening to the distal opening 109. The introducer sheath 100, or components thereof, may be formed by various materials, such as polymeric and/or metallic materials. In some instances, the introducer sheath 100, such as the elongate shaft of the introducer sheath 100, may include an additional surface coating, such as but not limited to, silicone, PET, or other applicable polymer.

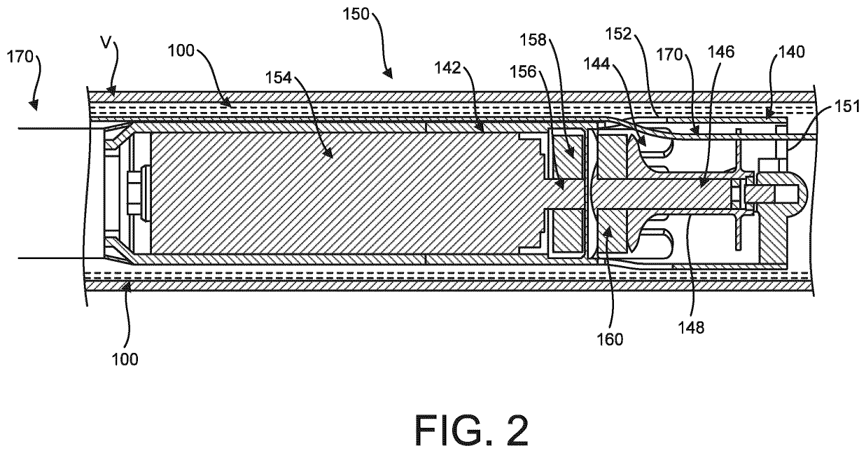

A hemostasis valve hub 120 (hereinafter “hub 120” for brevity) may be provided at the proximal end region 106 to provide access to the lumen 112 of the introducer sheath 100. The hub 120 may be configured for hemostasis by, for example, helping to prevent blood from leaking out of the introducer sheath 100 during use. For example, a medical device 170, such as a catheter or blood pump, may be inserted through the hub 120 and lumen 112 of the introducer sheath 100 and into the blood vessel V, and the hub 120 may maintain hemostasis between the medical device 170, the introducer sheath 100, and the external surroundings. In some embodiments, the medical device 170, may include and/or be coupled to a blood pump 150, shown in FIG. 2.

FIG. 2 illustrates a cross-sectional view of the body portion 110 of the introducer sheath 100 of FIG. 1 upon insertion of a medical device, illustratively a blood pump 150, into the introducer sheath 100. As noted above, the medical device 170 of FIG. 1 may be coupled to or include the blood pump 150, with the medical device 170 extending outside the blood vessel V and the introducer sheath 100. The blood pump 150 may be advanced through the blood vessel V and positioned in a target location, such as a target cardiac location (e.g., the left ventricle), via the introducer sheath 100. The blood pump 150 may generally include an impeller assembly housing 140 and a motor housing 142. In some embodiments, the impeller assembly housing 140 and the motor housing 142 may be integrally or monolithically constructed (e.g., formed as single unitary structure). In other instances, the impeller assembly housing 140 and the motor housing 142 may be separate components. The impeller assembly housing 140 carries an impeller assembly 144 therein. The impeller assembly 144 may include an impeller shaft 146 and an impeller 148 that rotate relative to the impeller assembly housing 140 to drive blood through the blood pump 150. More specifically, the rotation of the impeller 148 causes blood to flow from a blood inlet 151 formed on the impeller assembly housing 140, through the impeller assembly housing 140, and out of a blood outlet 152 formed on the impeller assembly housing 140. In some embodiments, the impeller shaft 146 and the impeller 148 may be integrally formed as a single unitary structure, whereas, in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may be coupled to a distally extending cannula, and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly 144. More specifically, in embodiments incorporating the impeller shaft 146, the impeller shaft 146 and the impeller 148 are configured to rotate with the driven magnet 160. In other embodiments, the motor 154 may be coupled to the impeller assembly 144 via other components. While the introducer sheath 100 is illustrated above with the use of the blood pump 150, various other medical devices may be used in conjunction with the introducer sheath 100 and the hemostasis valve hub 120.

In some embodiments, the introducer sheath 100 may be removed from the body after insertion of the medical device 170. It is contemplated that the introducer sheath 100 may be a splittable sheath which allows the introducer sheath 100 to be torn or broken apart along a predefined weakened area. For example, a splittable sheath may include one or more regions of weakness extending along a longitudinal axis thereof which allows the introducer sheath 100 to be removed from the body and peeled away from the medical device 170 such that the introducer sheath 100 does not remain in the body for an entire duration of the medical intervention. Similarly, the hub 120 may also be removed from the medical device 170. In some cases, the hub 120 may be a snap on hub which allows the hub 120 to be removed from the medical device 170 and/or the introducer sheath 100. In some examples, the introducer sheath 100 may be similar in form and function to the sheath 200 described herein and the hub 120 may be similar in form and function to the hub 300 described herein.

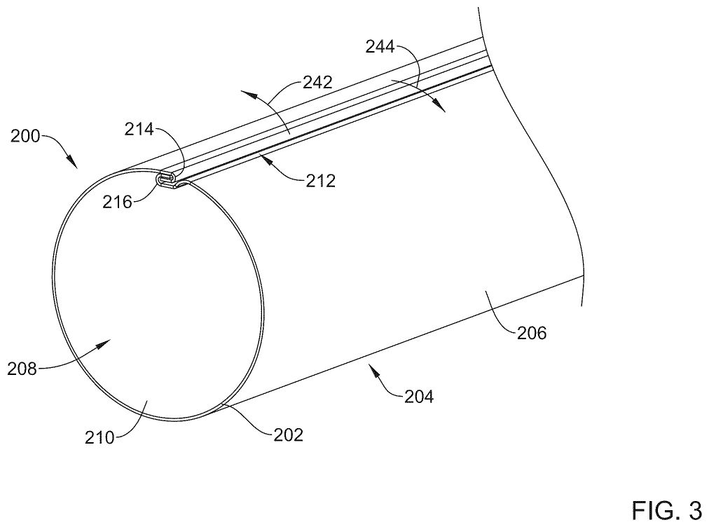

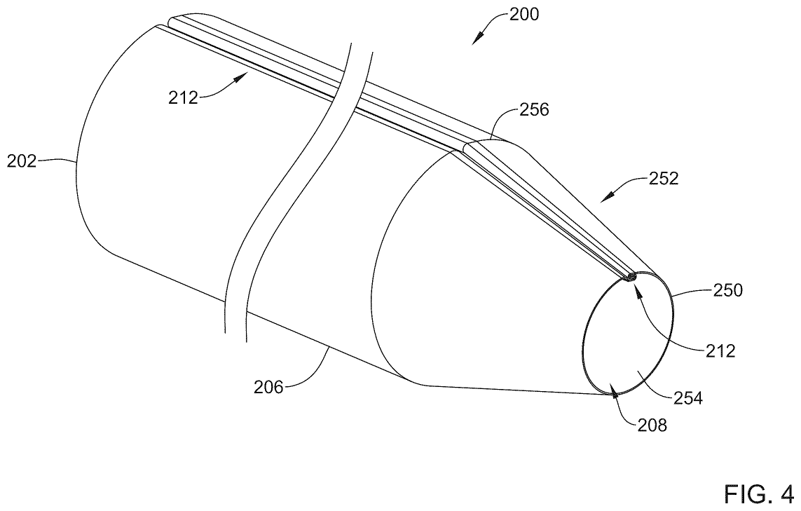

Once the medical intervention is complete, it may be desirable to use a removal sheath to remove the medical device 170 to protect the vasculature from damage that may be caused by removing a bare medical device 170. FIG. 3 is a proximal perspective view of a portion an illustrative sheath 200 that may be positioned over a medical device 170 that is already positioned within the body and FIG. 4 is a distal perspective view of the illustrative sheath 200. The sheath 200 may extend from a proximal end region 204 proximate a proximal end 202 of the sheath 200 to a distal end region 252 proximate a distal end 250 of the sheath 200 that is opposite the proximal end region 204. A body portion or elongate shaft 206 of the sheath 200 extends between the proximal end region 204 and the distal end region 252, and the body portion 206 defines a lumen 208 of the sheath 200. The sheath 200 includes a proximal opening 210 adjacent the proximal end region 204 and a distal opening 254 adjacent the distal end region, with the lumen 208 extending from the proximal opening 210 to the distal opening 254. The sheath 200, or components thereof, may be formed by various materials, such as polymeric and/or metallic materials. In some instances, the sheath 200, such as the elongate shaft 206 of the sheath 200, may include an additional surface coating, such as but not limited to, silicone, PET, or other applicable polymer.

In some embodiments, an outer diameter of the sheath 200 may vary along a length thereof. For example, the distal end region 252 may taper or reduce in diameter from an intermediate location 256 proximal to the distal end 250 to the distal end 250. The inner diameter may taper in a similar manner. It is contemplated that tapering the distal end region 252 of the sheath 200 may allow for the gradual dilation of the vessel as the sheath 200 is inserted. The angle of the taper may vary to achieve the desired effect. In some cases, the distal end region 252 may be formed from a softer material than a remaining portion of the sheath 200 to provide a more atraumatic distal end region 252, although this is not required. In other embodiments, the sheath 200 may have a substantially constant outer diameter from the proximal end 202 to the distal end 252.

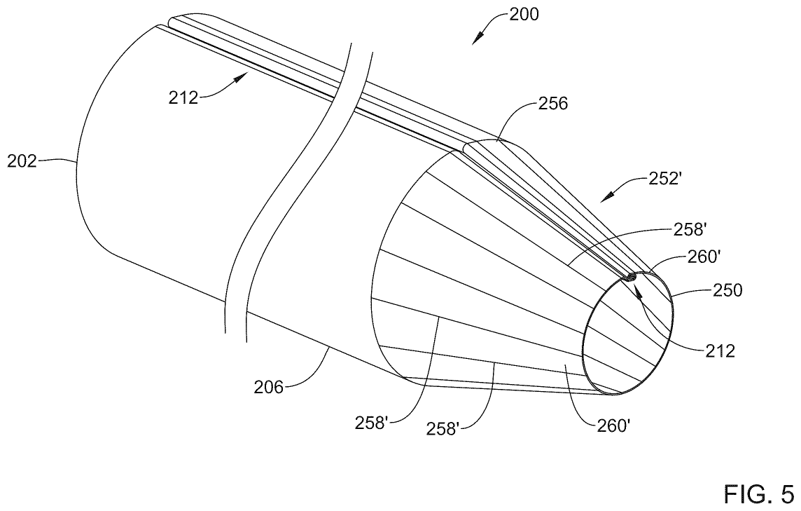

Referring briefly to FIG. 5, which illustrates a distal perspective view of the illustrative sheath 200 having an alternative distal end region 252′. In some cases, the distal end region 252′ may include a plurality of slits 258′ extending from the intermediate location 256 (or proximal end of the taper) to the distal end 250. The plurality of slits 258′ may extend through a thickness of a sidewall of the body portion 206 to define a plurality of fingers or flaps 260′. For brevity and ease of understanding, not every slit 258′ or every finger 260′ has be identified in FIG. 5. The slits 258′ may be spaced about a circumference of the distal end region 252′ in a uniform or non-uniform arrangement. The flaps 260′ may be configured to radially expand to accommodate a range of sizes of medical devices 170. For example, the flaps 260′ may be used with a medical device 170 having an outer diameter similar in size to the inner diameter of distal end 250 when the flaps 260′ are radially collapsed (e.g., in contact with the adjacent flaps 260′) to minimum inner diameter at the distal end 250, as shown in FIG. 5. In another example, the flaps 260′ may radially expand to accommodate a medical device 170 having an outer diameter similar in size to the inner diameter of the proximal end 202 of the sheath 200. It is contemplated that the medical device 170 may have an outer diameter anywhere between the minimum inner diameter of the distal end 250 with the flaps 260′ radially collapsed to the inner diameter of the proximal end 202 and the flaps 260′ in a maximum radially expanded configuration. When the flaps 260′ are in a radially expanded configuration a space or gap may be present between adjacent flaps 260′. In some embodiments, the flaps 260′ may be formed from a material having a similar stiffness as the medical device 170 such that the flaps 260′ can radially expand if necessary.

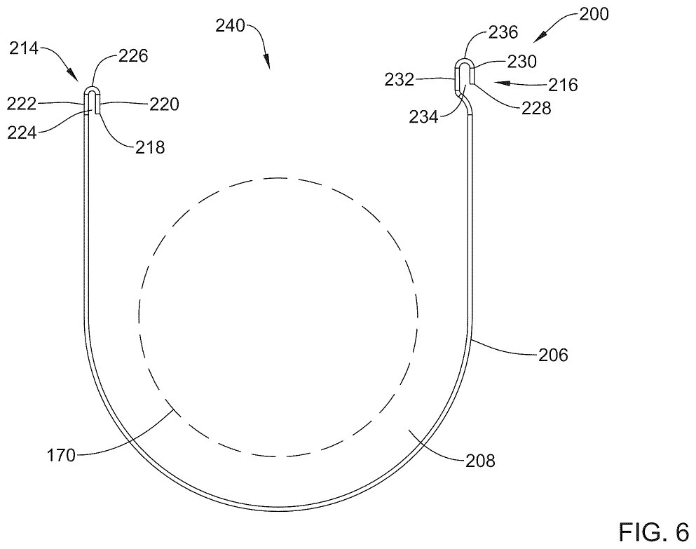

As shown in FIG. 3, the sheath 200 may include a hemostatic scam or connection 212 extending from the proximal end 202 to the distal end along a longitudinal axis thereof. The seam 212 may be a connection interface between a first connection member 214 and a second connection member 216. It is contemplated that the seam 212 may be fluid tight to prevent blood, or other fluid loss, via the seam 212. The first connection member 214 and the second connection member 216 may form a releasable interlock to hold the sheath 200 in a tubular configuration. For example, the sheath 200 may be moveable between the closed, tubular configuration shown in FIG. 3 and an open, channel configuration shown in FIG. 6, which illustrates an end view of the sheath 200 in the open configuration.

In the open channel configuration, the first connection member 214 and the second connection member 216 may be spaced from one another to define an opening 240 extending along a length of the elongate shaft 206 and configured to allow the sheath 200 to be positioned over another device. For example, the medical device 170 may pass laterally through the opening 240 and into the lumen 208 of the sheath 200. Once the medical device 170 is within the lumen 208, the first and second connection members 214, 216 may be coupled to one another.

The first connection member 214 and the second connection member 216 may each have a generally “U” or horseshoe shaped configuration, although this is not required. It is contemplated that the other geometric connections may be used, as desired. The first connection member 214 may have a free end 218. The free end 218 may be bent such that a region of the body portion 206 folds over on itself to form a first leg 220 extending generally parallel to a second leg 222. The first and second legs 220, 222 may be interconnected by a curved connection region 226. Further, the first and second legs 220, 222 may be spaced from one another by a gap or recess 224. Similarly, the second connection member 216 may have a free end 228. The free end 228 may be bent such that a region of the body portion 206 folds over on itself to form a first leg 230 extending generally parallel to a second leg 232. The first and second legs 230, 232 may be interconnected by a curved connection region 236. Further, the first and second legs 230, 232 may be spaced from one another by a gap or recess 234.

The first connection member 214 may be curved such that the first leg 220 extends towards or into the opening 240 while the second connection member 216 may be curved such that the first leg 230 thereof extends away from the opening 240. This configuration may allow the first leg 220 of the first connection member 214 to be received within the gap 234 of the second connection member 216 and the first leg 230 of the second connection member 216 to be received within the gap 224 of the first connection member 214 when the connection members 214, 216 are interconnected, as shown in FIG. 3. It is contemplated that when the connection members 214, 216 are interconnected the first connection member 214 may exert a pulling force in a first direction, as shown at arrow 242 and the second connection member 216 may exert a pulling force in a second direction opposite the first direction, as shown at arrow 244. The opposing pulling forces may compress the free ends 218, 228 of the connection members 214, 216 against an inner surface of the opposing curved regions 226, 236 to form a fluid tight seal. For example, the free end 218 of the first connection member 214 may compress against the inner surface (e.g., the surface within the gap 234) of the curved region 236 of the second connection member 216 and the free end 228 of the second connection member 216 may compress against the inner surface (e.g., the surface within the gap 224) of the curved region 226 of the first connection member 214. In some embodiments, an elastomeric material may be provided along the seam 212 to increase the scalability of the seam 212, although this is not required. For example, the connection members 214 may be formed from or include a layer of an elastomeric, deformable, or otherwise compliant material. Additionally, or alternatively, the seam 212 may include a chemical bond, such as, but not limited to a glue, adhesive, etc.

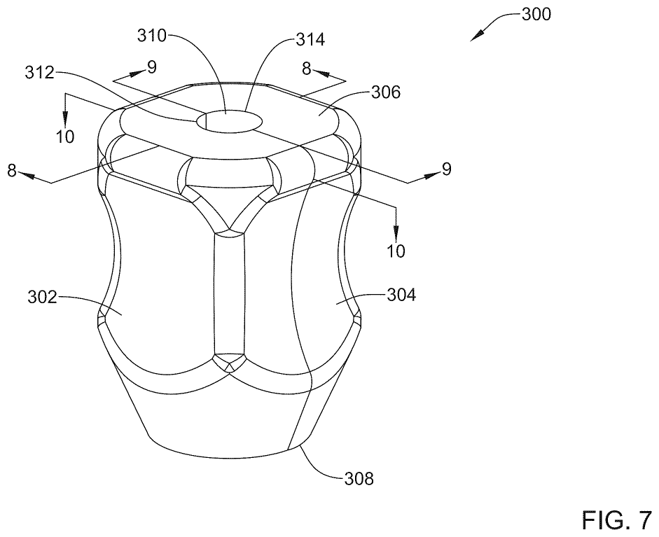

FIG. 7 is a perspective view of an illustrative removable hemostasis valve hub 300 (hereinafter “hub 300” for brevity). The hub 300 may be configured to be coupled with the sheath 200 after the sheath 200 has been positioned over the medical device 170 (which is at least partially deployed in the body). The hub 300 may include a first body portion 302 and a second body portion 304. The first and second body portions 302, 304 may be formed from separate structures that are releasably coupled to each other. In other examples, the first and second body portions 302, 304 may be formed as a single unitary structure that are coupled along a scam or hinge extending from a proximal end 306 to a distal end 308 thereof. The first and second body portions 302, 304 may extend from the proximal end 306 to the distal end 308 with a lumen 310 extending from the proximal end 306 to the distal end 308. The first body portion 302 may include a first recessed region 312 and the second body portion 304 may include a second recessed region 314. When the first and second body portions 302, 304 are coupled together, the recessed regions 312, 314 align to define the lumen 310.

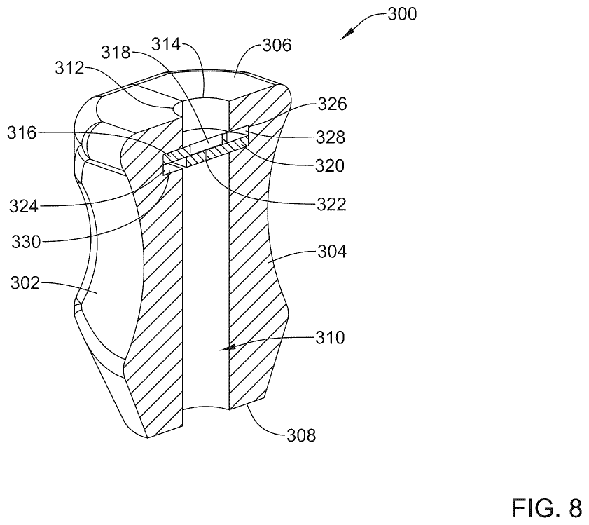

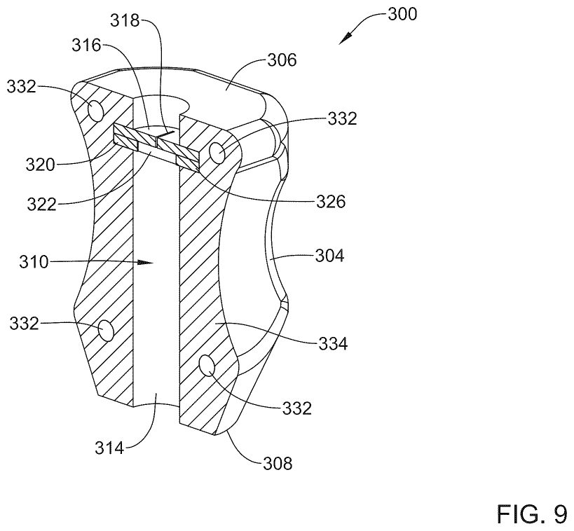

FIG. 8 is a cross-sectional view of the illustrative hub 300 taken at line 8-8 of FIG. 7. The first body portion 302 may include a semi-annular recess 324 configured to receive a first or proximal seal member 316 and a second or distal seal member 320. Similarly, the second body portion 304 may include a semi-annular recess 326 configured to receive the first seal member 316 and the second seal member 320. When the first body portion 302 is coupled with the second body portion 304, the semi-annular recesses 324, 326 may be aligned to form an annular recess extending radially from the lumen 310. The first seal member 316 may be secured to the first body portion 302. In some cases, a proximal surface and/or a side surface of the first seal member 316 may be secured within and/or to the semi-annular recess 324. The first seal member 316 may include a first slit 318 extending through a thickness thereof. The second seal member 320 may be secured to the second body portion 304. In some cases, a distal surface and/or a side surface of the second seal member 320 may be secured within and/or to the semi-annular recess 326. The second seal member 320 may include a second slit 322 extending through a thickness thereof, as shown in FIG. 9 which is a cross-sectional view of the illustrative hub 300 taken at line 9-9 of FIG. 7. The second slit 322 may extend generally orthogonal to the first slit 318. This may allow the medical device 170 to pass through the first and second seal members 316, 320 while providing a hemostatic seal. In some examples, one or both of the slits 318, 322 may extend to an outer edge of the sealing member 316, 320 to allow the hub 300 to be assembled over medical device 170 without having to insert the medical device 170 through the slits 318, 322 in a direction parallel to a longitudinal axis of the hub 300.

In some embodiments, the first and second seal members 316, 320 may be configured to overlap across the lumen 310. However, the first and second seal members 316, 320 may not each extend across an entirety of the annular recess created by the semi-annular recess 324. More particularly, the first seal member 316 may have a generally truncated circular cross-sectional shape such that a portion of the first seal member 316 extends into the semi-annular recess 326 of the second body portion 304 and a portion 328 of the semi-annular recess 326 is free from the first seal member 316. However, this is not required. In some cases, the cross-section of the first seal member 316 may be generally circular or may take other shapes as desired. Similarly, the second seal member 320 may have a generally truncated circular cross-sectional shape such that a portion of the second seal member 320 extends into the semi-annular recess 324 of the first body portion 302 and a portion 330 of the semi-annular recess 324 is free from the second seal member 320. However, this is not required. In some cases, the cross-section of the second seal member 320 may be generally circular or may take other shapes as desired. It is contemplated that positioning at least a portion of the first seal member 316 within the semi-annular recess 326 of the second body portion 304 and at least a portion of the second seal member 320 within the semi-annular recess 324 of the first body portion 302 (as shown in FIG. 9) may help prevent the seal members 316, 320 from deflecting along a longitudinal axis of the hub 300 as medical device 170 are inserted or withdrawn therethrough. Thus, the semi-annular recesses 324, 326 may have a height that is approximately equal to a combined height of the first and second sealing member 316, 320.

The first and second seal members 316, 320 may be formed from an elastomeric or deformable material, such as, but not limited to silicone. This may allow slits 318, 322 of the first and second seal members 316, 320 to expand around the medical device 170 when the medical device 170 is disposed within the lumen 310 while allowing the slits 318, 322 of the first and second seal members 316, 320 to return to an unexpanded configuration (as shown in FIGS. 8 and 9) upon removal of the medical device 170. It is contemplated that this may allow the hub 300 to be used to insert the medical device 170 into the body, be removed, and then reattached to the medical device 170 for removal of the medical device 170 from the body.

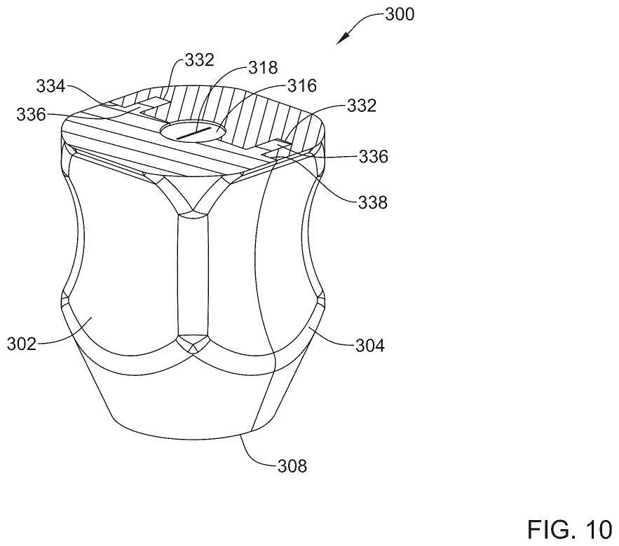

Referring to FIG. 9 and FIG. 10, which is a cross-sectional view of the illustrative hub 300 taken at line 10-10 of FIG. 7, the hub 300 may include features to releasably secure the first body portion 302 with the second body portion 304. In one example, the second body portion 304 may include a plurality of apertures 332 extending inward from a connection surface 334. While FIG. 9 shows the first body portion 302 as including four apertures 332, the first body portion 302 may include fewer than four apertures 332 (e.g., one or more) or more than four apertures 332, as desired. The first body portion 302 may include a plurality of posts or pins 336 extending outward from a connection surface 338 thereof. It is contemplated that the first body portion 302 may include a same number of pins 336 (e.g., one or more) as the second body portion 304 has apertures 332. When the first body portion 302 is coupled with the second body portion 304, the pins 336 of the first body portion 302 may be inserted into mating apertures 332 of the second body portion 304. In some examples, the pins 336 may form a friction fit with the apertures 332. Such a connection may releasably secure the first body portion 302 to the second body portion 304 while the connection precludes unintentional uncoupling of the first and second body portions 302, 304. Other connection mechanisms, such as, but not limited to, snap fits, etc. may be used, as desired. In some examples, the first and/or second body portions 302, 304 may include a recess adjacent to the connection surfaces 334, 338 along an outer surface thereof to allow a finger or tool to be used to help separate the first and second body portions 302, 304 when so desired.

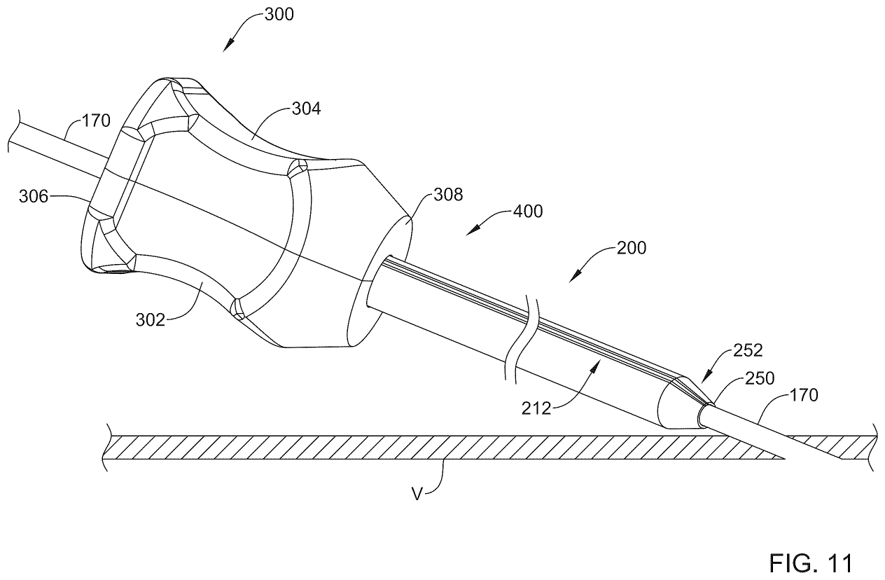

FIG. 11 is a schematic view of the sheath 200 and hub 300 disposed over a medical device 170 prior to removal of the medical device 170 with the vessel V in cross-section. As described above, it may be desirable to remove the insertion sheath and/or hub after insertion of the medical device 170 to reduce disruptions to blood flow. However, it may be desirable to use a sheath, such as sheath 200, to facilitate removal of the medical device 170 to reduce the likelihood of damage to the vessel V. To begin, the sheath 200 may be in the open, channel configuration with the first connection member 214 uncoupled from the second connection member 216, as shown in FIG. 6, and free from attachment to the hub 300. The sheath 200 may be placed around the medical device 170 with the medical device passing through the opening 240 such that the medical device 170 is disposed within the lumen 208 of the sheath 200. Next, the first connection member 214 may be coupled with the second connection member 216 along a length of the sheath 200 to form a tubular member along a length of the sheath 200. In one example, coupling the first connection member 214 with the second connection member 216 may include positioning the first leg 220 of the first connection member 214 within the gap 234 of the second connection member 216 and the first leg 230 of the second connection member 216 within the gap 224 of the first connection member 214. Positioning of the sheath 200 about the outer surface of the medical device 170 and coupling the first and second connection members 214, 216 may be performed with the sheath 200 external to the body.

Next, the hub 300 may be coupled to the proximal end region 204 of the sheath 200. In some examples, the first or second body portion 302, 304 may be positioned against an outer surface of the proximal end region 204 of the sheath 200. In some cases, the proximal end 202 of the sheath 200 may be configured to contact a distal surface of the second (or distal) sealing member 320, although this is not required. The second of the first or second body portion 302, 304 may then be secured to the first of the first or second body portion 302, 304 such that the proximal end region 204 of the sheath 200 is disposed within the lumen 310 of the hub 300. In other examples, the first and second body portions 302, 304 of the hub 300 may be coupled to one another proximal to the proximal end 202 of the sheath 200 and then distally advanced to engage the hub 300 with the proximal end region 204 of the sheath 200. It is contemplated that the hub 300 may form a friction fit, a press fit, a threaded engagement, a snap fit, or other mechanical engagement with the sheath 200. As the sheath 200 is disposed over the medical device 170 prior to coupling the hub 300 to the sheath 200, the hub 300 may be coupled to the medical device 170 while it is coupled being coupled to the sheath 200. As described herein, the medical device 170 may extend through the slits 318, 322 such that the seal members 316, 320 form a fluid tight or hemostatic seal about the outer surface of the medical device 170.

Once the hub 300 is coupled to the proximal end region 204 of the sheath 200, the hub and sheath assembly 400 may be distally advanced to position the distal end region 352 and an intermediate region of the sheath 200 within the vessel lumen. The medical device 170 may be proximally retracted though the sheath 200 until it is removed from the body. Next, the sheath 200 may be removed from the body.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A hub and sheath assembly, comprising:
a valve hub including a first body portion, a second body portion, and a lumen extending therethrough; and
an elongate shaft including a lumen and a seam, the lumen and the seam extending from a proximal end to a distal end of the elongate shaft, the elongate shaft releasably coupled to the valve hub;
wherein the seam is movable from an interlocked configuration configured to hold the elongate shaft in a generally tubular configuration and an open configuration defining an opening extending along a length of elongate shaft.

2. The hub and sheath assembly of claim 1, wherein the seam includes a first connection member and a second connection member.

3. The hub and sheath assembly of claim 2, wherein the first connection member and the second connection member are configured to form a releasable interlock.

4. The hub and sheath assembly of claim 2, wherein:
the first connection member comprises a first leg, a second leg extending generally parallel to the first leg, a curved region connecting the first leg and the second leg, and a gap between the first leg and the second leg; and
the second connection member comprises a first leg, a second leg extending generally parallel to the first leg, a curved region connecting the first leg and the second leg, and a gap between the first leg and the second leg.

5. The hub and sheath assembly of claim 4, wherein when the seam is in the interlocked configuration, the first leg of the first connection member is disposed within the gap of the second connection member and the first leg of the second connection member is disposed within the gap of the first connection member.

6. The hub and sheath assembly of claim 1, wherein a distal end region of the elongate shaft tapers towards the distal end thereof.

7. The hub and sheath assembly of claim 1, wherein a distal end region of the elongate shaft includes a plurality of flaps, the plurality of flaps configured to radially expand.

8. The hub and sheath assembly of claim 1, wherein the first body portion is releasably coupled to the second body portion.

9. The hub and sheath assembly of claim 1, further comprising a first sealing member disposed within the lumen of the hub.

10. The hub and sheath assembly of claim 9, further comprising a second sealing member disposed within the lumen of the hub.

11. The hub and sheath assembly of claim 9, wherein the first sealing member comprises a slit extending through a thickness thereof.

12. The hub and sheath assembly of claim 11, wherein the second sealing member comprises a slit extending through a thickness thereof, the slit of the second sealing member extending generally orthogonal to the slit of the first sealing member.

13. The hub and sheath assembly of claim 10, wherein the first sealing member is coupled to the first body portion and the second sealing member is coupled to the second body portion.

14. The hub and sheath assembly of claim 1, wherein the first body portion includes one or more pins extending from a connection surface thereof and the second body portion includes one or more apertures extending into the second body portion from a connection surface thereof.

15. The hub and sheath assembly of claim 14, wherein the one or more pins are configured to be releasably coupled with the one or more apertures.

16. A hub and sheath assembly, comprising:
a valve hub comprising:
a first body portion;
a second body portion releasably coupled to the first body portion;
a lumen extending from a proximal end to a distal end of the valve hub; and
a first sealing member disposed within the lumen of the valve hub; and
an elongate shaft having a lumen and a seam including a first connection member and a second connection member, the lumen and the seam extending from a proximal end to a distal end of the elongate shaft, the elongate shaft releasably coupled to the valve hub;
wherein the seam is movable from an interlocked configuration configured to hold the elongate shaft in a generally tubular configuration and an open configuration defining an opening extending along a length of elongate shaft.

17. The hub and sheath assembly of claim 16, wherein a distal end region of the elongate shaft tapers towards the distal end thereof.

18. A method for removing a medical device from a body lumen, the method comprising:
positioning an elongate sheath having an opening extending along a length thereof over a portion of the medical device exterior to the body lumen;
securing a first connection member and a second connection member of the elongate sheath along the length of the elongate sheath to form a tubular member;
coupling a hemostasis valve hub to a proximal end region of the elongate sheath to form a valve hub and sheath assembly;
distally advancing the hemostasis valve hub and sheath assembly over the medical device and into the body lumen; and
proximally retracting the medical device from the body lumen through the hemostasis valve hub and sheath assembly.

19. The method of claim 18, wherein coupling the hemostasis valve hub to the proximal end region of the elongate sheath comprises coupling a first body portion of the hemostasis valve hub to a second body portion of the hemostasis valve hub over the medical device at a location proximal to a proximal end of the sheath and distally advancing the hemostasis valve hub over the proximal end of the sheath.

20. The method of claim 18, wherein the first connection member and the second connection member form a hemostatic seal.
